(19) United States
(12) Patent Application Publication
(10) Pub. No.: US 2026/0218053 A1
(43) Pub. Date: Jul. 30, 2026

(54) SURFACTANT COMPOSITIONS DERIVED FROM WASTE STREAMS, AND METHODS

(71) Applicant: Kemira Oyj, Helsinki (FI)

(72) Inventors: Erkki Johannes Metsälä, Helsinki (FI); Viktor Kukkonen, Espoo (FI); Jussi Nikkarinen, Helsinki (FI)

(21) Appl. No.: 19/146,513

(22) PCT Filed: Jan. 19, 2024
(86) PCT No.: PCT/US2024/012184

§ 371 Date: Jul. 8, 2025

Related U.S. Application Data
(60) Provisional application No. 63/480,773, filed on Jan. 20, 2023.
(60) Provisional application No. 63/587,298, filed on Oct. 2, 2023.

(51) Int. Cl. C09K 23/18; C07C 213/08; C07C 227/08; C09K 23/00

(52) U.S. Cl. CPC C09K 23/18; C07C 213/08; C07C 227/08; C09K 23/018

(57) ABSTRACT

Compositions, such as surfactant compositions, and methods of forming compositions, such as surfactant compositions, which may include a gemini surfactant. The methods may use a starting material from a waste stream from another process.

### CROSS-REFERENCE TO RELATED APPLICATIONS

This application claims priority to U.S. Provisional Patent Application No. 63/480,773, filed Jan. 20, 2023, and U.S. Provisional Patent Application No. 63/587,298, filed Oct. 2, 2023, which are incorporated by reference herein.

### TECHNICAL FIELD

The present disclosure generally relates to surfactants, including surfactants derived from one or more waste streams, compositions that include one or more surfactants, and methods for producing surfactants.

### BACKGROUND

Gemini surfactants usually include two surfactant molecules chemically bonded together by a spacer. The two terminal hydrocarbon tails of gemini surfactants can be short (e.g., 5 to 15 carbon atoms) or long (e.g., 16 to 30 carbon atoms); the two polar head groups of a gemini surfactant can be cationic, anionic, or nonionic; and the spacer can be short (e.g., 1 to 3 carbon atoms), long (e.g., 4 to 6 carbon atoms), flexible, or rigid, and/or may contain one or more heteroatoms (e.g., nitrogen, oxygen, sulfur, phosphorus, etc.).

Moreover, components of a gemini surfactant may or may not be symmetrically disposed about the center of the spacer. Some gemini surfactants can self-assemble at much lower concentrations, and/or have improved surface activity compared to conventional surfactants. Due to these options and features, gemini surfactants can be tailored for a number of applications, and/or useful in a number of applications.

For example, gemini surfactants may be useful in catalysis and adsorption applications, as new synthetic vectors for gene transfection, in analytical separations, in solubilization processes, in nanoscale technologies, in biotechnologies, in enhanced oil recovery, and/or as additives in a number of products, such as paint.

There remains a need for methods of producing surfactants, including gemini surfactants, that are cost-effective, use starting materials that are readily available and/or inexpensive, or a combination thereof. There also remains a need for compositions, including surfactant compositions, that include a gemini surfactant.

### BRIEF SUMMARY

Provided herein are compositions, which may include a gemini surfactant, and methods of forming compositions, such as surfactant compositions, which may use readily available and/or relatively inexpensive starting materials, such as starting materials that are present in a waste stream from other processes.

In one aspect, methods of forming compositions, such as surfactant compositions, are provided. In some embodiments, the methods include (i) providing a starting material that includes methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxy propanoate, 2-(dimethylamino)ethyl 3-(2-(dimethylamino)ethoxy)propanoate, 2-(dimethylamino)ethyl acrylate, 2-(dimethylamino)ethan-1-ol, or a combination thereof;

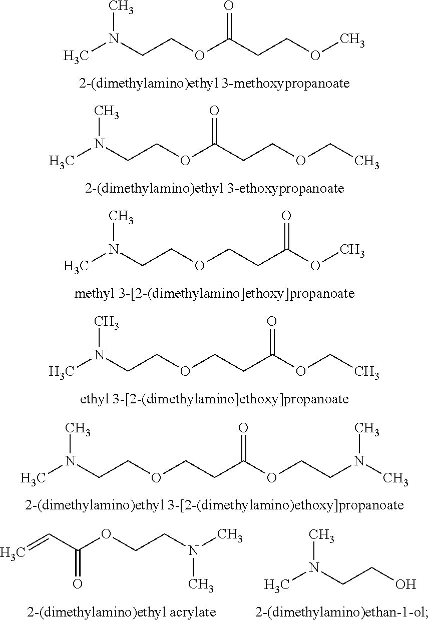

and (ii) contacting the starting material with an alkylation agent to form an alkylated material. The providing of the starting material may include providing a stream, such as a waste stream, and separating from the stream at least a portion of the methyl 3-(2-(dimethylamino)-ethoxy)propanoate, at least a portion of the ethyl 3-(2-(dimethylamino)-ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof.

In another aspect, compositions, such as surfactant compositions, are provided. In some embodiments, the compositions include a compound of formula (I), a compound of formula (II), a compound of formula (III), a compound of formula (IV), a compound of formula (V), a compound of formula (VI), a compound of formula (VII), or a compound of formula (VIII), or a combination thereof, and, additionally or alternatively, any one or more products obtained by a hydrolysis, such as an ester hydrolysis, of the compound of formula (I), the compound of formula (II), the compound of formula (III), the compound of formula (IV), the compound of formula (V), the compound of formula (VI), the compound of formula (VII), the compound of formula (VIII), or the combination thereof:

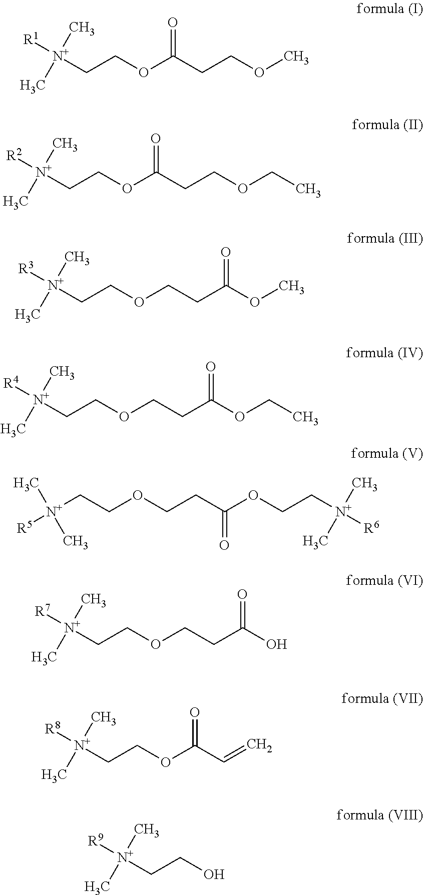

wherein R¹, R², R³, R⁴, R⁵, R⁶, R⁷, R⁸, and R⁹ are independently selected from the group consisting of a C₅-C₃₀ hydrocarbyl, formula (a), formula (b), and formula (c);

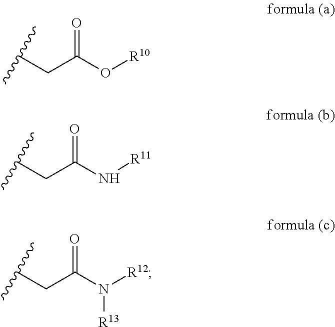

and wherein R¹⁰ and R¹¹, independently, are a C₅-C₃₀ hydrocarbyl, and R¹² and R¹³, independently, are a C₁-C₃₀ hydrocarbyl.

Additional aspects will be set forth in part in the description which follows, and in part will be obvious from the description, or may be learned by practice of the aspects described herein. The advantages described herein may be realized and attained by means of the elements and combinations particularly pointed out in the listing of embodiments and the appended claims. It is to be understood that both the foregoing general description and the following detailed description are exemplary and explanatory only and are not restrictive.

### BRIEF DESCRIPTION OF THE DRAWINGS

FIG. 1 is a plot of surface tension in deionized water at about 24° C. versus concentration (mmol) for an embodiment of a surfactant composition.

FIG. 2 is a plot of surface tension in deionized water at about 24° C. versus concentration (ppm) for an embodiment of a surfactant composition.

### DETAILED DESCRIPTION

Provided herein are methods of making compositions and compositions, such as surfactant compositions.

### Compositions

In some embodiments, the compositions include a compound of formula (I), a compound of formula (II), a compound of formula (III), a compound of formula (IV), a compound of formula (V), a compound of formula (VI), a compound of formula (VII), a compound of formula (VIII), or a combination thereof, and, additionally or alternatively, any one or more products obtained by a hydrolysis, such as an ester hydrolysis, of the compound of formula (I), the compound of formula (II), the compound of formula (III), the compound of formula (IV), the compound of formula (V), the compound of formula (VI), the compound of formula (VII), the compound of formula (VIII), or the combination thereof.

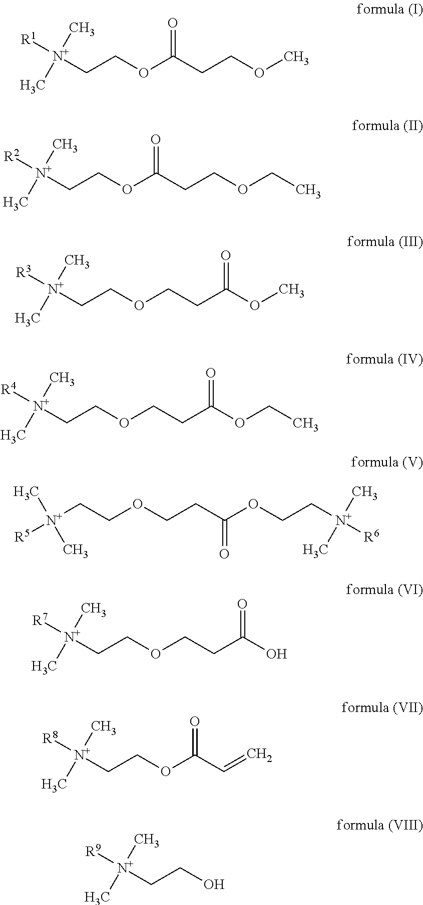

wherein R¹, R², R³, R⁴, R⁵, R⁶, R⁷, R⁸, and R⁹ are independently selected from the group consisting of a C₅-C₃₀ hydrocarbylm formula (a), formula (b), and formula (c):

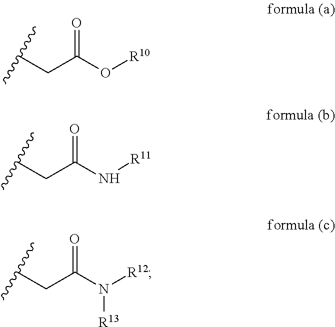

and
wherein R¹⁰ and R¹¹, independently, are a C₅-C₃₀ hydrocarbyl, and R¹² and R¹³, independently, are a C₁-C₃₀ hydrocarbyl.

In some embodiments, the compositions include a compound of formula (I), a compound of formula (II), a compound of formula (III), a compound of formula (IV), a compound of formula (V), a compound of formula (VI), or a combination thereof, and, additionally or alternatively, any one or more products obtained by a hydrolysis, such as an ester hydrolysis, of the compound of formula (I), the compound of formula (II), the compound of formula (III), the compound of formula (IV), the compound of formula (V), the compound of formula (VI), or the combination thereof:

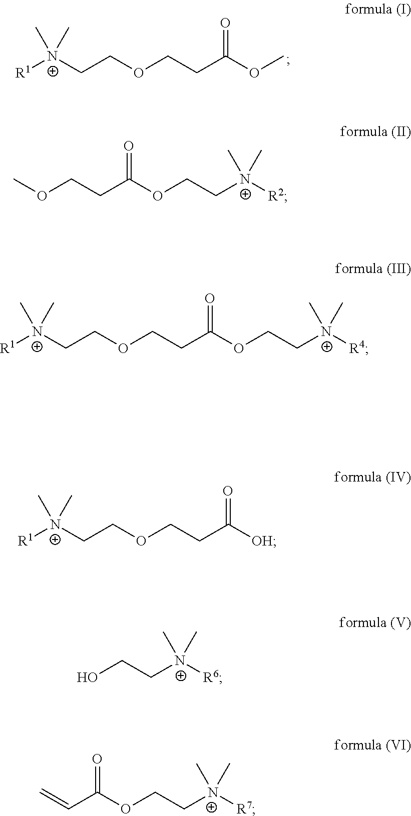

wherein R¹, R², R³, R⁴, R⁵, R⁶, and R⁷ are independently selected from the group consisting of a C₅-C₃₀ hydrocarbyl, formula (a), and formula (b);

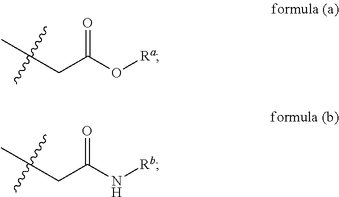

and
wherein R^{a} and R^{b}, independently, are a C₅-C₃₀ hydrocarbyl.

When the formulas or compounds depicted herein include a positively charged moiety or atom, such as a positively charged quaternary nitrogen atom, the formulas or compounds may include any counteranion. In some embodiments, the compound of formula (I), the compound of formula (II), the compound of formula (III), the compound of formula (IV), the compound of formula (V), and the compound of formula (VI), the compound of formula (VII), the compound of formula (VIII), include a counteranion corresponding to each quaternary nitrogen atom. The counteranions may include inorganic ions, such as a halide ion, or organic ions, such as an alkyl sulfonate or an aryl sulfonate.

In some embodiments, R¹, R², R³, R⁴, R⁵, R⁶, R⁷, R⁸, R⁹, R¹⁰, R¹¹, R¹², R¹³, R^{a}, and R^{b}, independently, are a C₆-C₃₀ hydrocarbyl, C₆-C₂₈ hydrocarbyl, C₆-C₂₆ hydrocarbyl, C₆-C₂₄ hydrocarbyl, C₁₀-C₂₄ hydrocarbyl, C₁₀-C₂₀ hydrocarbyl, C₁₄-C₂₄ hydrocarbyl, C₁₀-C₁₇ hydrocarbyl, or a C₁₈-C₂₄ hydrocarbyl. The number of carbon atoms of a hydrocarbyl may be selected to impart one or more desirable characteristics to a composition or a component thereof. When the compositions described herein include a compound of formula (III), R³ and R⁴ may be the same or different. For example, (i) R³ may be a branched dodecyl group, and R⁴ may be a linear dodecyl group; (ii) R³ may be a branched dodecyl group, and R⁴ may be a branched hexyl group: (iii) R³ and R⁴ may be identical linear decyl groups; etc.

In some embodiments, the compositions include a compound of formula (III). The compound of formula (III) may have a structure according to formula (IIIa):

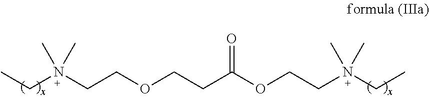

wherein x is 4 to 29.

In some embodiments, the compound of formula (III) has a structure according to formula (IIIb):

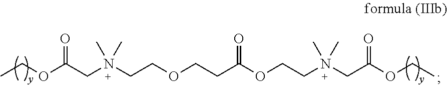

wherein y is 4 to 29.

In some embodiments, the compound of formula (III) has a structure according to formula (IIIc):

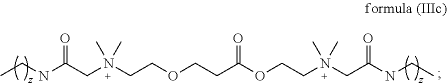

wherein z is 4 to 29. Although a linear alkyl chain is depicted in formula (IIIa), formula (IIIb), and formula (IIIc), non-linear C₅-C₃₀ hydrocarbyl groups are described herein.

In some embodiments, the compound of formula (III) has a structure according to formula (IIId):

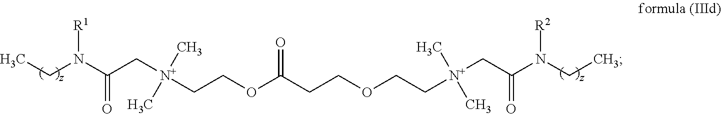

wherein z is 4 to 29. Although a linear alkyl chain is depicted in formula (IIIa), formula (IIIb), formula (IIIc), and formula (IIId), non-linear C₅-C₃₀ hydrocarbyl groups are described herein and R¹ and R² are linear or non-linear C₁-C₃₀ hydrocarbyl groups.

In some embodiments, the compositions include a compound of formula (III), and the compositions have a critical micelle concentration that is at least 6 times, at least 8 times, at least 10 times, at least 12 times, or at least 14 times less than a comparative critical micelle concentration of a comparative composition that includes a single-cation surfactant. For comparison purposes, the critical micelle concentration is measured at a temperature of about 20° C. to about 25° C.

In some embodiments, the compositions include a compound of formula (III), and, with regard to corrosion inhibition or biological inhibition, the compositions have a minimal inhibitory concentration that is at least 6 times, at least 8 times, at least 10 times, at least 12 times, or at least 14 times less than a comparative minimal inhibitory concentration of a comparative composition that includes a single-cation surfactant.

A compound of formula (I), a compound of formula (II), a compound of formula (III), a compound of formula (IV), a compound of formula (V), a compound of formula (VI), a compound of formula (VII), a compound of formula (VIII), or a combination thereof may be present at any concentration in the compositions provided herein. For example, a compound of formula (I), a compound of formula (II), a compound of formula (III), a compound of formula (IV), a compound of formula (V), a compound of formula (VI), a compound of formula (VII), a compound of formula (VIII), or a combination thereof may be present in the composition at a concentration of at least 50%, at least 60%, at least 70%, at least 80%, at least 90%, at least 95%, or at least 99%, by weight, based on the weight of the composition. Therefore, if a composition having a weight of 100 units includes 30 units of a compound of formula (III) and 20 units a compound of formula (IV), then the composition includes 50%, by weight, of the combination of compounds of formulas (III) and (IV). As a further example, a compound of formula (I), a compound of formula (II), a compound of formula (III), a compound of formula (IV), a compound of formula (V), a compound of formula (VI), a compound of formula (VII), a compound of formula (VIII), or a combination thereof may be present in a composition at a concentration of about 0.01% to about 25%, about 0.01% to about 20%, about 0.01% to about 15%, about 0.01% to about 10%, about 0.01% to about 7.5%, about 0.01% to about 5%, about 0.01% to about 3%, about 0.01% to about 2%, or about 0.01% to about 1%, by weight, based on the weight of the composition. The compositions described herein may be a detergent, a cosmetic, a personal care product, a coating (e.g., a paint), a biocide, a pharmaceutical formulation, an anti-corrosion agent, an enhanced oil recovery fluid, a fabric softener, a leveling agent, a drying agent, a solubilizer, an emulsifier, a dispersant, a foaming agent, a mining fluid, a waste water treatment fluid, an anti-foaming agent, an anti-static agent, or a combination thereof. The compositions described herein may be an additive for a detergent, a cosmetic, a personal care product, a coating (e.g., a paint), a biocide, a pharmaceutical formulation, an anti-corrosion agent, an enhanced oil recovery fluid, a fabric softener, a leveling agent, a drying agent, a solubilizer, an emulsifier, a dispersant, a foaming agent, a mining fluid, a waste water treatment fluid, an anti-foaming agent, an anti-static agent, or a combination thereof. Other uses and applications, however, are envisioned.

### Methods of Forming Compositions

Also provided herein are methods of forming compositions, such as surfactant compositions. In some embodiments, the methods include providing a starting material that includes methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxy propanoate. 2-(dimethylamino)ethyl 3-(2-(dimethylamino)ethoxy)propanoate, 2-(dimethylamino)ethyl acrylate, 2-(dimethylamino)ethan-1-ol, or a combination thereof:

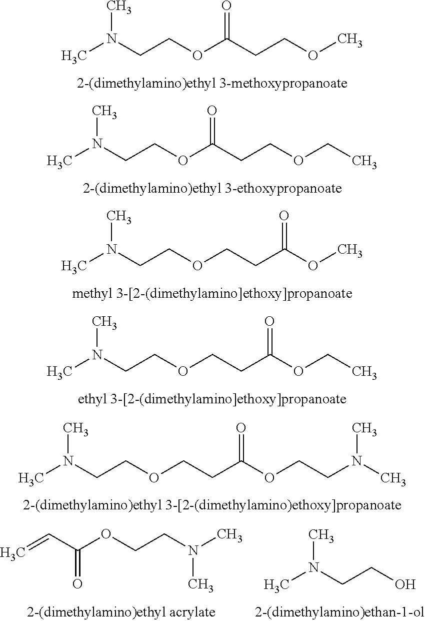

Any one or more components of the starting material may be obtained from any source. In some embodiments, the providing of the starting material includes (i) providing a stream that includes methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxypropanoate, 2-(dimethylamino)ethyl 3-(2-(dimethylamino)ethoxy)propanoate, 2-(dimethylamino)ethyl acrylate. 2-(dimethylamino)ethan-1-ol, methyl 3-methoxypropanoate, or a combination thereof,

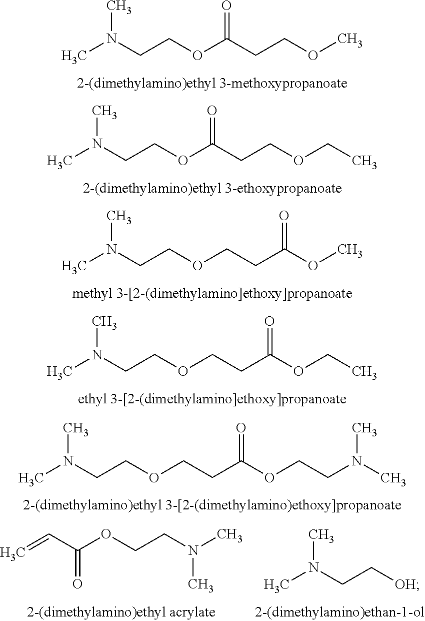

and
(ii) separating from the stream at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof. The separating of compounds from the stream may be achieved using any known technique. In some embodiments, the one or more compounds is separated from a stream via distillation or rectification.

The stream that includes methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxypropanoate, 2-(dimethylamino)ethyl 3-(2-(dimethylamino)ethoxy)propanoate, 2-(dimethylamino)ethyl acrylate, 2-(dimethylamino)ethan-1-ol, methyl 3-methoxypropanoate, ethyl 3-ethoxypropanoate or a combination thereof may be a waste stream. The waste stream may be from a process that produces a polymerizable monomer or a precursor thereof, such as an acrylate monomer or a methacrylate monomer.

In some embodiments, the methods also include converting at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol or a combination thereof to 2-(dimethylamino)ethyl 3-(2-(dimethylamino)ethoxy)propanoate. The 2-(dimethylamino)ethyl 3-(2-(dimethylamino)-ethoxy)propanoate may then be processed as described herein, such as by contacting it with an alkylation agent, subjecting it to hydrolysis, or a combination thereof. The converting of the at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof to 2-(dimethylamino)ethyl 3-(2-(dimethylamino)ethoxy) propanoate may be achieved in any manner. In some embodiments, the converting of the at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof to 2-(dimethylamino)ethyl 3-(2-(dimethylamino)ethoxy) propanoate includes contacting (i) the at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof, and (ii) a base. The base may be present at any effective amount, such as a catalytic amount. The base may include any of those known in the art, such as a hydroxide, e.g., NaOH, KOH, etc. The contacting may occur in a liquid. The liquid may be any liquid that does not undesirably impact the conversion. The liquid may include an aqueous liquid and/or an organic liquid. The liquid may include an alcohol, such as an amino alcohol. An “amino alcohol” is a compound that includes at least one alcohol functional group and at least one amine; an example of an amino alcohol is 2-(dimethylamino)ethan-1-ol.

The streams, such as waste streams, that may be subjected to the methods described herein may include various concentrations of one or more of the starting materials and other components. In some embodiments, methyl 3-(2-(dimethylamino)ethoxy)propanoate is present in the stream at a concentration of about 1% to about 20%, about 8% to about 20%, or about 10% to about 15%, by weight. In some embodiments, 2-(dimethylamino)ethyl 3-methoxypropanoate is present in the stream at a concentration of about 1% to about 60%, about 10% to about 40%, about 15% to about 30%, or about 20% to about 25%, by weight. In some embodiments, 2-(dimethylamino)ethyl 3-(2-(dimethylamino)ethoxy)propanoate is present in the stream at a concentration of about 5% to about 40%, about 10% to about 40%, about 20% to about 40%, or about 25% to about 35%, by weight. In some embodiments, 2-(dimethylamino)ethyl acrylate is present in the stream at a concentration of about 1% to about 25%, about 10% to about 20%, or about 12% to about 18%, by weight. In some embodiments, methyl 3-methoxypropanoate is present in the stream at a concentration of about 0.5 to about 2%, or about 0.8% to about 1.2%, by weight. In some embodiments, 2-(dimethylamino)ethyl acrylate is present in the stream at a concentration of about 1% to about 25%, about 5% to about 20%, or about 10% to about 20%, by weight.

The starting materials may be contacted with an alkylation agent to form an alkylated material. The alkylation agent may include any of those known in the art, and generally is a compound that is capable of covalently bonding a hydrocarbyl group, directly or via a functional group (such as an amide or ester), to a compound of formula (I), a compound of formula (II), a compound of formula (III), a compound of formula (IV), a compound of formula (V), a compound of formula (VI), a compound of formula (VII), a compound of formula (VIII), or a combination thereof. In some embodiments, the alkylation agent includes—

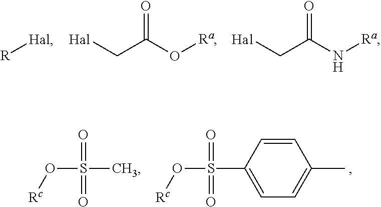

or a combination thereof;
wherein Hal is a halogen atom, such as Cl, Br, or I; and wherein R, R^{a}, R^{b}, R^{c}, and R^{d}, independently, are a C₅-C₃₀ hydrocarbyl. In some embodiments, R, R^{a}, R^{b}, R^{c}, and R^{d}, independently, are a C₆-C₃₀ hydrocarbyl, C₆-C₂₈ hydrocarbyl, C₆-C₂₆ hydrocarbyl, C₆-C₂₄ hydrocarbyl, C₁₀-C₂₄ hydrocarbyl, C₁₀-C₂₀ hydrocarbyl, C₁₄-C₂₄ hydrocarbyl, C₁₀-C₁₇ hydrocarbyl, or a C₁₈-C₂₄ hydrocarbyl. R, R^{a}, R^{b}, R^{c}, and R^{d} may be selected to impart a desirable property to a composition, such as a desirable hydrophilic-lipophilic balance (HLB).

In some embodiments, the alkylation agent includes—

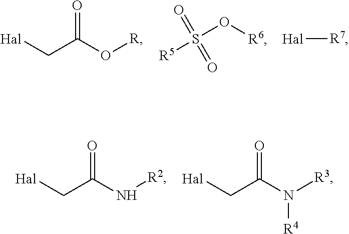

or a combination thereof;
wherein Hal is a halogen atom, such as Cl, Br, or I; and wherein R, R², R³, R⁴, R⁵, R⁶, and R⁷ independently, are a C₅-C₃₀ hydrocarbyl. In some embodiments, R, R², R³, R⁴, R⁵, R⁶, and R⁷, independently, are a C₆-C₃₀ hydrocarbyl, C₆-C₂₈ hydrocarbyl, C₆-C₂₆ hydrocarbyl, C₆-C₂₄ hydrocarbyl, C₁₀-C₂₄ hydrocarbyl, C₁₀-C₂₀ hydrocarbyl, C₁₄-C₂₄ hydrocarbyl, C₁₀-C₁₇ hydrocarbyl, or a C₁₈-C₂₄ hydrocarbyl. R, R², R³, R⁴, R⁵, R⁶, and R⁷ may be selected to impart a desirable property to a composition, such as a desirable hydrophilic-lipophilic balance (HLB).

A starting material generally may be contacted with any amount of an alkylation agent. In some embodiments, a starting material is contacted with an amount of moles of the alkylation agent that equals or exceeds an amount of moles of tertiary nitrogen atoms in the starting material. In some embodiments, a starting material is contacted with an amount of moles of the alkylation agent that is less than an amount of moles of tertiary nitrogen atoms in the starting material. An amount of alkylation agent that is used may be selected to impart a desirable property to a composition, such as a desirable hydrophilic-lipophilic balance (HLB).

An alkylated material may be subjected to further reaction, such as a hydrolysis reaction to produce a hydrolyzed material. The hydrolysis reaction may be an ester hydrolysis reaction, which may cleave an ester functional group as depicted in the following scheme:

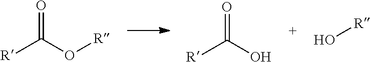

The hydrolysis reaction, such as an ester hydrolysis reaction, may include contacting an alkylated material and water, wherein the water has a pH less than 7 or greater than 7, and the pH is effective to achieve hydrolysis, such as ester hydrolysis. Therefore, a hydrolysis reaction may be a base catalyzed hydrolysis reaction or an acid catalyzed hydrolysis reaction. In some embodiments, the alkylated material includes a compound of formula (III), a compound of formula (IIIa), a compound of formula (IIIb), a compound of formula (IIIc), a compound of formula (IIId), or a combination thereof;

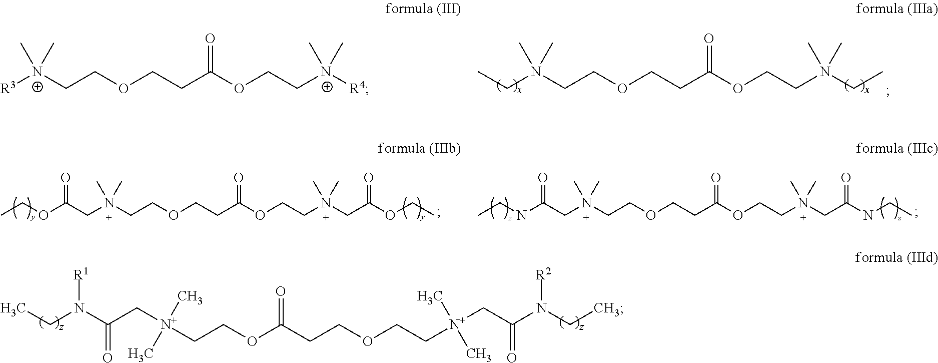

wherein x, y, and z, independently, are 4 to 29; and R¹ and R², independently, are a C₁-C₃₀ hydrocarbyl; R³ and R⁴, independently, are a C₅-C₃₀ hydrocarbyl; and wherein the hydrolyzed material includes a compound of formula (III′), a compound of formula (III″), a compound of formula (IIIa′), a compound of formula (IIIa″), a compound of formula (IIIb′), a compound of formula (IIIb″), a compound of formula (IIIc′), a compound of formula (IIIc″), a compound of formula (IIId′), a compound of formula (IIId″), or a combination thereof;

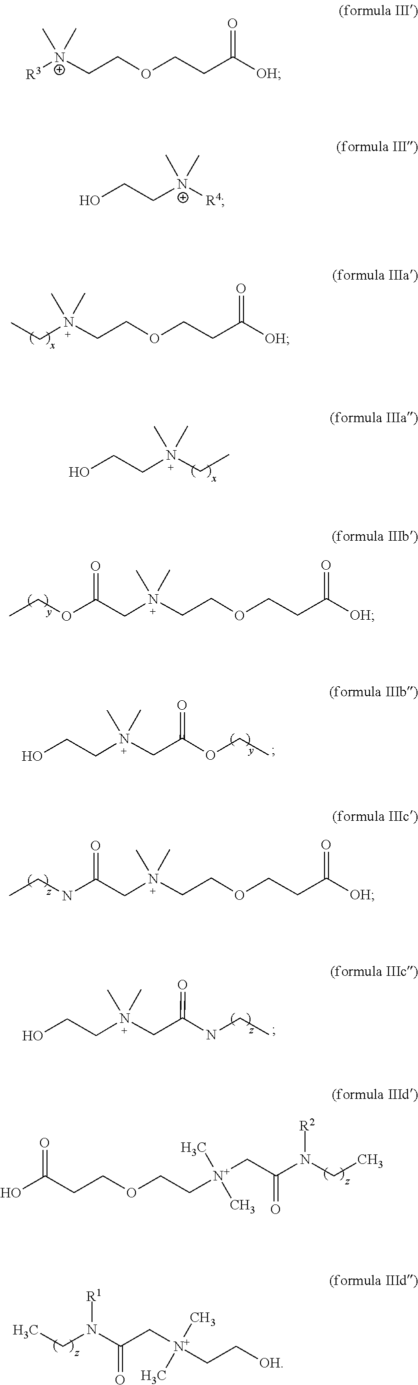

When used herein with regard to the selection of a substituent (for example, any of the various “R” groups), the term “independently” indicates that (i) a substituent at a particular location may be the same or different for each molecule a formula (e.g., (i) a compound of formula (I) may include two molecules of formula (I), with each molecule having the same or a different C₅-C₃₀ hydrocarbyl selected for R¹; or (ii) two differently labeled substituents selected from the same group of substituents may be the same or different (e.g., R³ and R⁴ of formula (III) may both be selected from “a C₁₀-C₂₄ hydrocarbyl”, and the C₁₀-C₂₄ hydrocarbyls selected for R³ and R⁴ may be the same or different).

The phrases “C₁-C₃₀ hydrocarbyl,” “C₁₀-C₂₄ hydrocarbyl”, and the like, as used herein, generally refer to aliphatic, aryl, or arylalkyl groups containing 1 to 30 carbon atoms, or 10 to 24 carbon atoms, respectively, including substituted derivatives thereof. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and/or linear analogs or derivatives thereof, in each instance having, for example, 1 to 30 total carbon atoms or 10 to 24 total carbon atoms for a “C₁-C₃₀ hydrocarbyl” and “C₁₀-C₂₄ hydrocarbyl”, respectively. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl. Cycloalkyl moieties may be monocyclic or multicyclic, and examples include cyclopropyl, cyclobutyl, cyclopentyl, cyclohexyl, and adamantyl, including any heteroatom substituted derivative thereof. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl. 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, anthracenyl, tolyl, xylyl, mesityl, benzyl, and the like, including any heteroatom substituted derivative thereof.

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ hydrocarbyl may include, but is not limited to, a pyrimidinyl moiety, a pyridinyl moiety, a dioxanyl moiety, a diethyl ether moiety, a methyl propionate moiety, an N,N-dimethylacetamide moiety, a butoxy moiety, etc., and a “substituted” aryl C₁₂ hydrocarbyl may include, but is not limited to, an oxydibenzene moiety, a benzophenone moiety, etc.) or (ii) one or more of its hydrogen atoms (e.g., chlorobenzene may be characterized generally as an aryl C₆ hydrocarbyl “substituted” with a chlorine atom) is substituted with a chemical moiety or functional group such as acyl, alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), primary, secondary, and tertiary amino (such as alkylamino, arylamino, arylalkylamino), aryl, arylalkyl, aryloxy, azo, azido, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, cycloalkyl, cycloalkenyl, ester, ether (e.g., methoxy, ethoxy), halo, haloalkyl (e.g., —CCl₃, —CF₃, —C(CF₃)₃), haloalkoxy, trihalomethanesulfonyl, trihalomethanesulfonamido, heteroalkyl, heterocycloalkyl, heteroaryl, heteroarylalkyl, isocyanate, isothiocyanate, nitrile, nitro, oxo, phosphodiester, silyl, sulfide, sulfonamido (e.g., SO₂NH₂), sulfone, sulfenyl, sulfinyl, sulfonyl (including alkylsulfonyl, arylsulfonyl and arylalkylsulfonyl), sulfoxide, thiocarbonyl, thiocarbamyl, thiocyanato, thiol (e.g., sulfhydryl, thioether) or urea (—NHCONH-alkyl-).

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is.” “containing,” “having,” and “comprises” are used in an open-ended fashion, and thus should be interpreted to mean “including, but not limited to.” When compositions or methods are claimed or described in terms of “comprising” various steps or components, the compositions or methods can also “consist essentially of” or “consist of” the various steps or components, unless stated otherwise.

The terms “a,” “an,” and “the” are intended to include plural alternatives, e.g., at least one. For instance, the disclosure of “a stream”, “an alkylation agent”, and the like, is meant to encompass one, or mixtures or combinations of more than one stream, alkylation agent, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, that one of several “R” groups may be a C₁₀-C₂₄ hydrocarbyl. This range should be interpreted as encompassing a C₁₀ hydrocarbyl and a C₂₄ hydrocarbyl, and the further encompasses each of a C₁₁, C₁₂, C₁₃, C₁₄, C₁₅, C₁₆, C₁₇, C₁₈, C₁₉, C₂₀, C₂₁. C₂₂, and C₂₃ hydrocarbyl, including any ranges and sub-ranges between any of these values.

As used herein, the term “about” means plus or minus 10% of the numerical value of the number with which it is being used.

### EXAMPLES

The present disclosure is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present disclosure or the scope of the appended claims. Thus, other aspects of this disclosure will be apparent to those skilled in the art from consideration of the specification and practice of the aspects disclosed herein.

### Example 1—Preparation of an Embodiment of a Surfactant

A round bottom reaction flask was charged with 8.35 g (about 35.5 mmol) of 2-(dimethylamino)ethyl 3-(2-(dimethylamino)ethoxy)propanoate, and 18.72 g (about 70 mmol) of dodecyl 2-chloroacetate (n=9).

The reaction mixture was stirred at 50° C. for one hour, and this temperature was maintained for 20 hours, without stirring. The reaction likely proceeded according to the following scheme (n=9):

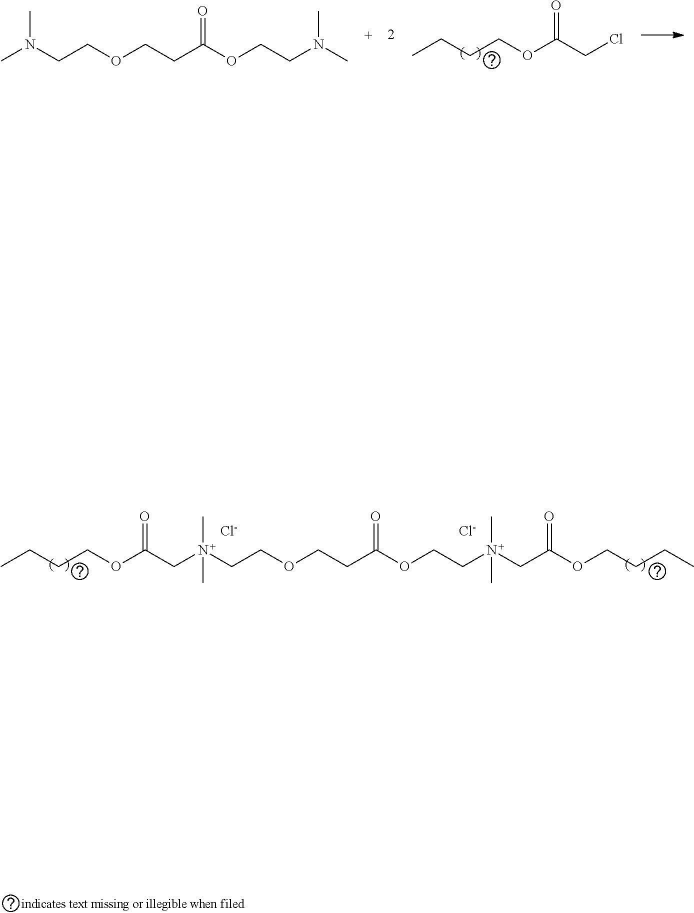

The product N,N′-(((1-oxopropane-1,3-diyl)bis(oxy))bis(ethane-2,1-diyl))bis(2-(dodecyloxy)-N,N-dimethyl-2-oxoethan-1-aminium) dichloride was a colorless waxy solid.

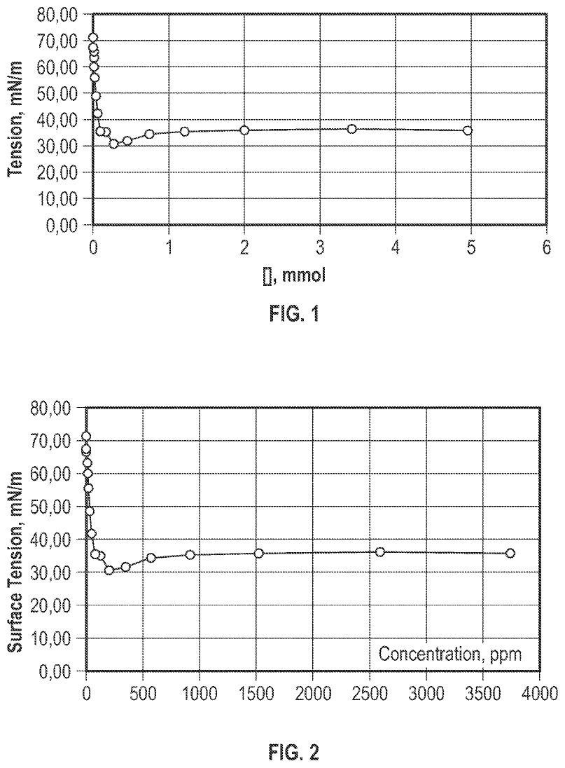

The product was characterized with ¹H NMR, and the surface tension of the product was determined in deionized water at about 24° C. at various concentrations. These results are provided at FIG. 1 and FIG. 2. The critical micelle concentration of the product was about 0.07 mmol to about 0.1 mmol.

### Example 2—Preparation of an Embodiment of a Surfactant

A round bottom reaction flask was charged with 11.02 g (about 47 mmol) of 2-(dimethylamino)ethyl 3-(2-(dimethylamino)ethoxy)propanoate, and 36.92 g (about 110 mmol) of 1-bromooctadecane, and 50 mL acetone.

The reaction mixture was stirred and refluxed for 72 hours at 70° C. The resulting reaction mixture was a colorless crystal dispersion. The precipitate was filtered, washed with 100 mL of acetone, and refluxed in 100 mL of acetone.

The reaction likely proceeded according to the following scheme (n=16):

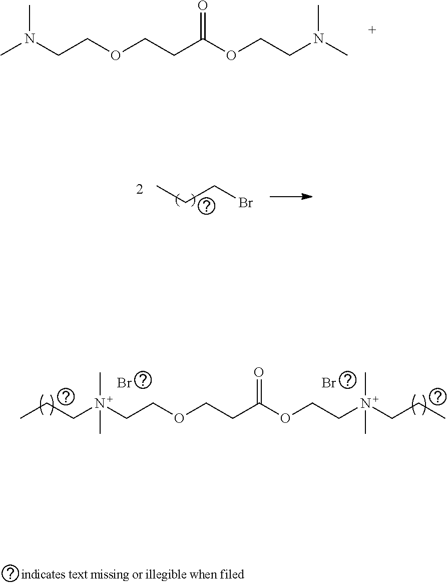

After cooling to room temperature, the colorless precipitate was filtered, washed with 100 mL of acetone, and dried at room temperature in vacuo.

The reaction yield was about 75.7%. The product was N,N′-(((1-oxopropane-1,3-diyl)bis(oxy))bis(ethane-2,1-diyl))bis(N,N-dimethyloctadecan-1-aminium) dibromide. The product was characterized with 1H NMR.

### Example 3—Preparation of an Embodiment of a Surfactant

A round bottom reaction flask was charged with 12.07 g (about 52 mmol) of 2-(dimethylamino)ethyl 3-(2-(dimethylamino) ethoxy) propanoate, and 35.0 g (about 100 mmol) of octadecyl 2-chloroacetate.

The reaction mixture was stirred at 50° C. for one hour, and this temperature was maintained for 20 hours without stirring. The reaction likely proceeded according to the following scheme (n=15):

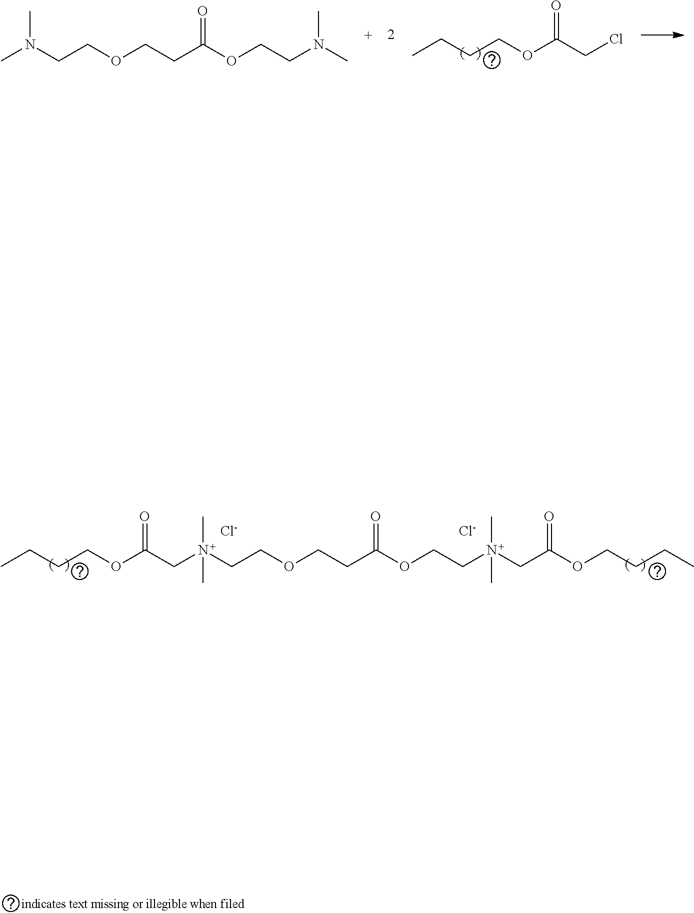

The product was N,N′-(((1-oxopropane-1,3-diyl)bis(oxy))bis(ethane-2,1-diyl))bis(2-(octadecyloxy)-N,N-dimethyl-2-oxoethan-1-aminium) dichloride, and was a colorless brittle solid.

A portion of the product (about 5.12 g) was extracted twice with 50 ml of acetonitrile, while stirring at 50° C. The first extraction lasted 3 hours, and the second extraction occurred over night. The product, after extraction, was a colorless powder, which was dried under reduced pressure (about 20 mbar) at room temperature for 5 hours. The products were characterized with ¹H NMR.

### Example 4—Conversion of Mixed Monoamines

In this example, the following abbreviations are used—

| Abbreviation | Compound |
|---|---|
| ADAME | 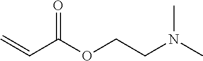 |
| | 2-(dimethylamino)ethyl acrylate |
| MEPAME | 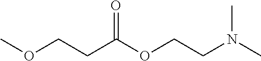 |
| | 2-(dimethylamino)ethyl 3-methoxypropanoate |
| a-MEPAME (anti-MEPAME) | 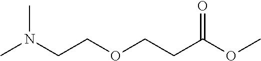 |
| | methyl 3-(2-(dimethylamino)ethoxy)propanoate |
| DMETOADAME | 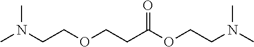 |
| | 2-(dimethylamino)ethyl 3-(2- |
| | dimethylamino)ethoxy)propanoate |
| DMAE | 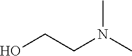 |
| | 2-(dimethylamino)ethan-1-ol |

In this example, the mixed monoamines subjected to conversion were obtained as a distillation fraction of a waste stream. The starting material included the following monoamines:

| Monoamine | wt % |
|---|---|
| DMAE | 2.39 |
| ADAME | 6.15 |
| MEPAME | 74.51 |
| a-MEPAME | 5.01 |
| DMETOADAME | 11.24 |

In this example, 78.6 g of the foregoing mixed monoamines were placed in a round bottom flask, and 2.5 g of potassium hydroxide in 37.5 g of DMAE were added to the round bottom flask in one portion. The 78.6 g of the mixed monoamines included the following amounts of the monoamines:

| Monoamine | Mass (g) | Moles |
|---|---|---|
| DMAE | 1.88 | 0.0211 |
| ADAME | 4.83 | 0.0338 |
| MEPAME | 58.56 | 0.334 |
| a-MEPAME | 3.94 | 0.0225 |
| DMETOADAME | 8.83 | 0.0380 |

The resulting reaction mixture was stirred under reduced pressure at about 40 to about 50 mbar at 40° C. for about 2 hours. The reactions of this example were believed to proceed according to the following scheme:

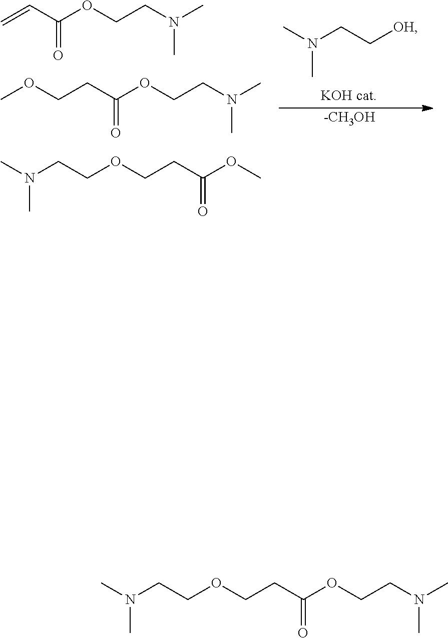

The reaction mixture was cooled to room temperature, and neutralized by adding 8 g of AMBERLYST® 15 ion exchange resin (DuPont de Nemours, Inc., USA) in H± form. After the ion exchange resin was separated by filtration, the product was analyzed by gas chromatography-flame ionization detection (GC-FID).

The results of the analysis are depicted at the following table:

| Component | Starting Material (wt %) | Product (wt %) |
|---|---|---|
| DMAE | 2.39 | 29.09 |
| ADAME | 6.15 | 2.52 |
| MEPAME | 74.51 | 39.1 |
| a-MEPAME | 5.01 | 2.96 |
| DMETOADA | 11.24 | 16.71 |

The data of this table demonstrates the successful conversion of a portion of the monoamines to DMETOADA, because the wt % of ADAME, MEPAME, and a-MEPTAME was less in the Product (due to their conversion and the addition of DMAE), and the wt % of DMETOADA was greater in the Product, despite the addition of DMAE. In summary, the data revealed that the conversion of monoamines to DMETOADAMA, in this example, was greater than 60%.

### Embodiments

The following is a listing of non-limiting embodiments.

Embodiment 1(A). A composition, such as a surfactant composition, comprising, consisting essentially of, or consisting of a compound of formula (I), a compound of formula (II), a compound of formula (III), a compound of formula (IV), a compound of formula (V), a compound of formula (VI), or a combination thereof, and, additionally or alternatively, any one or more products obtained by a hydrolysis, such as an ester hydrolysis, of the compound of formula (I), the compound of formula (II), the compound of formula (III), the compound of formula (IV), the compound of formula (V), the compound of formula (VI), the compound of formula (VII), the compound of formula (VIII), or the combination thereof:

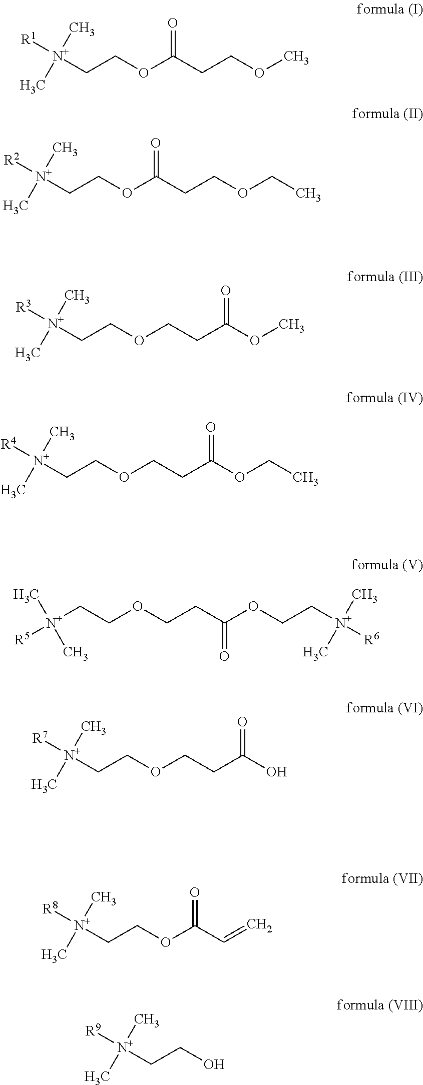

wherein R¹, R², R³, R⁴, R⁵, R⁶, R⁷, R⁸ and R⁹ are independently selected from the group consisting of a C₅-C₃₀ hydrocarbyl, formula (a), and formula (b), formula (c);

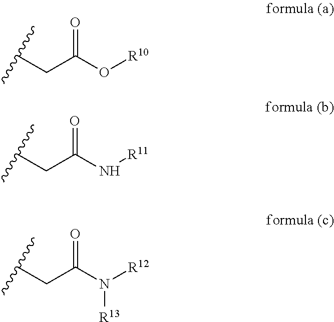

wherein R¹⁰ and R¹¹, independently, are a C₅-C₃₀ hydrocarbyl, and R¹² and R¹³, independently, are a C₁-C₃₀ hydrocarbyl; and/or R¹, R², R³, R⁴, R⁵, R⁶, R⁷, R⁸ and R⁹, independently, are derived from one or more of the following alkylation agents:

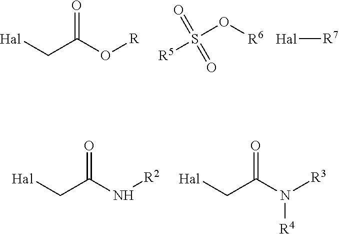

Embodiment 1(B). A composition, such as a surfactant composition, comprising, consisting essentially of, or consisting of a compound of formula (I), a compound of formula (II), a compound of formula (III), a compound of formula (IV), a compound of formula (V), a compound of formula (VI), or a combination thereof, and, additionally or alternatively, any one or more products obtained by a hydrolysis, such as an ester hydrolysis, of the compound of formula (I), the compound of formula (II), the compound of formula (III), the compound of formula (IV), the compound of formula (V), the compound of formula (VI), or the combination thereof:

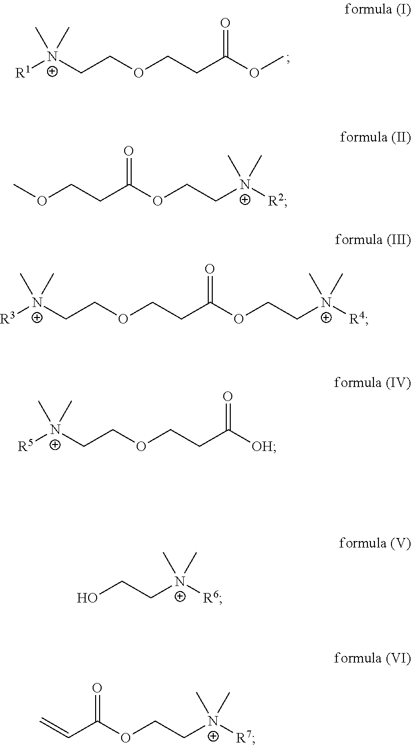

wherein R¹, R², R³, R⁴, R⁵, R⁶, and R⁷ are independently selected from the group consisting of a C₅-C₃₀ hydrocarbyl, formula (a), and formula (b):

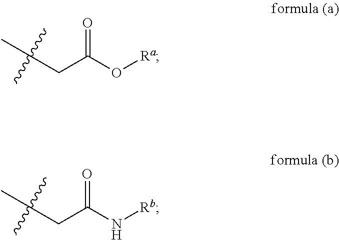

and
wherein R^{a} and R^{b}, independently, are a C₅-C₃₀ hydrocarbyl.

Embodiment 2. The composition of Embodiment 1, wherein the compound of formula (I), the compound of formula (II), the compound of formula (III), the compound of formula (IV), the compound of formula (V), the compound of formula (VI) the compound of formula (VII) and the compound of formula (VIII) comprise a counteranion corresponding to each quaternary nitrogen atom.

Embodiment 3. The composition of Embodiment 2, wherein the counteranion comprises an inorganic ion, for example, a halide ion, such as Cl⁻ or Br⁻.

Embodiment 4. The composition of Embodiment 2, wherein the counteranion comprises an organic ion, such as an alkyl sulfonate (for example, methane sulfonate), or an aryl sulfonate (for example, toluene sulfonate).

Embodiment 5A. The composition of any of the preceding embodiments, wherein R¹, R², R³, R⁴, R⁵, R⁶, R⁷, R⁸, R⁹, R¹⁰, R¹¹, R¹², R¹³, R^{a} and R^{b}, independently, are a C₆-C₃₀ hydrocarbyl, a C₆-C₂₈ hydrocarbyl, a C₆-C₂₆ hydrocarbyl, a C₆-C₂₄ hydrocarbyl, a C₁₀-C₂₄ hydrocarbyl, a C₁₀-C₂₀ hydrocarbyl, a C₁₄-C₂₄ hydrocarbyl, a C₁₀-C₁₇ hydrocarbyl, or a C₁₈-C₂₄ hydrocarbyl.

Embodiment 5B. The composition of any of the preceding embodiments, wherein R¹, R², R³, R⁴, R⁵, R⁶, R⁷, R^{a}, and R^{b}, independently, are a C₆-C₃₀ hydrocarbyl, a C₆-C₂₈ hydrocarbyl, a C₆-C₂₆ hydrocarbyl, a C₆-C₂₄ hydrocarbyl, a C₁₀-C₂₄ hydrocarbyl, a C₁₀-C₂₀ hydrocarbyl, a C₁₄-C₂₄ hydrocarbyl, a C₁₀-C₁₇ hydrocarbyl, or a C₁₈-C₂₄ hydrocarbyl.

Embodiment 6. The composition of any of the preceding embodiments, wherein the compound of formula (III) has a structure according to formula (IIIa):

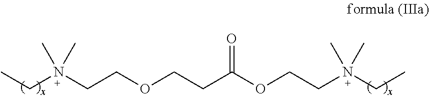

wherein x is 4 to 29.

Embodiment 7. The composition of any of the preceding embodiments, wherein the compound of formula (III) has a structure according to formula (IIIb):

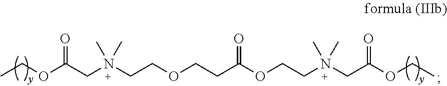

wherein y is 4 to 29.

Embodiment 8. The composition of any of the preceding embodiments, wherein the compound of formula (III) has a structure according to formula (IIIc):

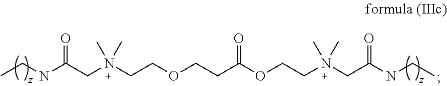

wherein z is 4 to 29; or
wherein the compound of formula (III) has a structure according to formula (IIId):

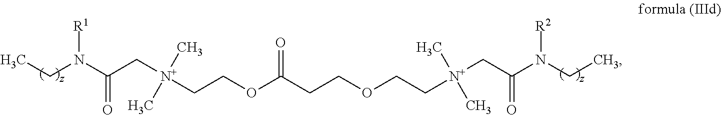

wherein z, independently, is 4 to 29 and R¹, R² are, independently, a C₁-C₃₀ hydrocarbyl.

Embodiment 9. The composition of any of the preceding embodiments, wherein the composition comprises, consists essentially of, or consists of the compound of formula (III), and the composition has a critical micelle concentration that is at least 6 times, at least 8 times, at least 10 times, at least 12 times, or at least 14 times less than a comparative critical micelle concentration of a comparative composition comprising a single-cation surfactant.

Embodiment 10. The composition of any of the preceding embodiments, wherein, with regard to corrosion inhibition or biological inhibition, the composition comprises, consists essentially of, or consists of the compound of formula (III), and the composition has a minimal inhibitory concentration that is at least 6 times, at least 8 times, at least 10 times, at least 12 times, or at least 14 times less than a comparative minimal inhibitory concentration of a comparative composition comprising a single-cation surfactant.

Embodiment 11. The composition of any of the preceding embodiments, wherein the compound of formula (I), the compound of formula (II), the compound of formula (III), the compound of formula (IV), the compound of formula (V), the compound of formula (VI), the compound of formula (VII), the compound of formula (VIII), or the combination thereof is present in the composition at a concentration of at least 50%, at least 60%, at least 70%, at least 80%, at least 90%, at least 95%, or at least 99%, by weight, based on the weight of the composition.

Embodiment 12. The composition of any of Embodiments 1 to 11, wherein the compound of formula (I), the compound of formula (II), the compound of formula (III), the compound of formula (IV), the compound of formula (V), the compound of formula (VI), the compound of formula (VII), the compound of formula (VIII), or the combination thereof is present in the composition at a concentration of about 0.01% to about 25%, about 0.01% to about 20%, about 0.01% to about 15%, about 0.01% to about 10%, about 0.01% to about 7.5%, about 0.01% to about 5%, about 0.01% to about 3%, about 0.01% to about 2%, or about 0.01% to about 1%, by weight, based on the weight of the composition.

Embodiment 13. The composition of Embodiment 12 or 13, wherein the composition is—or is an additive for—a detergent, a cosmetic, a personal care product, a coating (e.g., a paint), a biocide, a pharmaceutical formulation, an anti-corrosion agent, an enhanced oil recovery fluid, a fabric softener, a leveling agent, a drying agent, a solubilizer, an emulsifier, a dispersant, a foaming agent, a mining fluid, a waste water treatment fluid, an anti-foaming agent, an anti-static agent, or a combination thereof.

Embodiment 14A. A method of forming a composition, such as a surfactant composition, the method comprising (i) providing a starting material comprising, consisting essentially of, or consisting of methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxypropanoate, 2-(dimethylamino)ethyl 3-(2-(dimethylamino)ethoxy)propanoate. 2-(dimethylamino)ethyl acrylate, 2-(dimethylamino)ethan-1-ol, or a combination thereof;

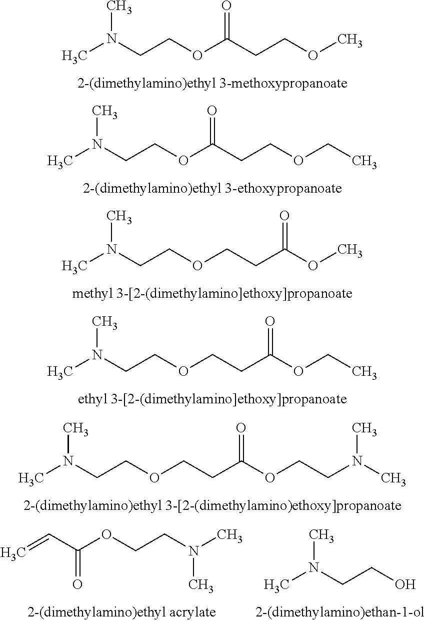

and
(ii) contacting the starting material with an alkylation agent to form an alkylated material.

Embodiment 14B. A method of forming a composition, such as a surfactant composition, the method comprising (i) providing a starting material comprising, consisting essentially of, or consisting of methyl 3-(2-(dimethylamino)ethoxy)propanoate, 2-(dimethylamino)ethyl 3-methoxypropanoate, 2-(dimethylamino)ethyl 3-(2-(dimethylamino)ethoxy)propanoate, 2-(dimethylamino)ethyl acrylate, 2-(dimethylamino)ethan-1-ol, or a combination thereof;

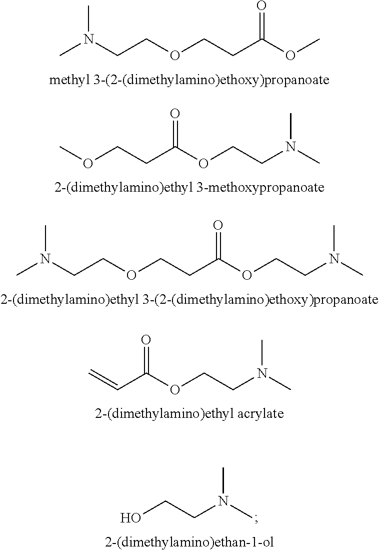

and
(ii) contacting the starting material with an alkylation agent to form an alkylated material.

Embodiment 15A. The method of Embodiment 14, wherein the alkylation agent comprises, consists essentially of, or consists of—

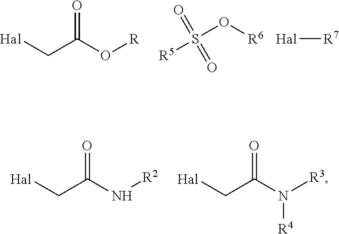

or a combination thereof;
wherein Hal is a halogen atom, such as Cl, Br, or I; and wherein R, R², R³, R⁶, and R⁷, independently, are a C₅-C₃₀ hydrocarbyl and R⁴ is C₁-C₃₀ hydrocarbyl.

Embodiment 15B. The method of Embodiment 14, wherein the alkylation agent comprises, consists essentially of, or consists of—

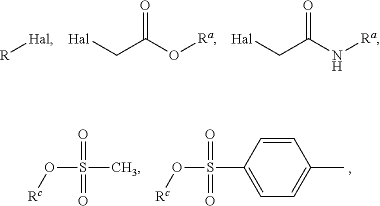

or a combination thereof;
wherein Hal is a halogen atom, such as Cl, Br, or I; and wherein R, R^{a}, R^{b}, R^{c}, and R^{d}, independently, are a C₅-C₃₀ hydrocarbyl.

Embodiment 16A. The method of Embodiment 16, wherein R, R², R³, R⁶, and R⁷, independently, are a C₆-C₃₀ hydrocarbyl, a C₆-C₂₈ hydrocarbyl, a C₆-C₂₆ hydrocarbyl, a C₆-C₂₄ hydrocarbyl, a C₁₀-C₂₄ hydrocarbyl, a C₁₀-C₂₀ hydrocarbyl, a C₁₄-C₂₄ hydrocarbyl, a C₁₀-C₁₇ hydrocarbyl, or a C₁₈-C₂₄ hydrocarbyl and R⁴ is C₁-C₃₀ hydrocarbyl.

Embodiment 16B. The method of Embodiment 15, wherein R, R^{a}, R^{b}, R^{c}, and R^{d}, independently, are a C₆-C₃₀ hydrocarbyl, a C₆-C₂₈ hydrocarbyl, a C₆-C₂₆ hydrocarbyl, a C₆-C₂₄ hydrocarbyl, a C₁₀-C₂₄ hydrocarbyl, a C₁₀-C₂₀ hydrocarbyl, a C₁₄-C₂₄ hydrocarbyl, a C₁₀-C₁₇ hydrocarbyl, or a C₁₈-C₂₄ hydrocarbyl.

Embodiment 17. The method of any of Embodiments 14 to 16, wherein the starting material is contacted with an amount of moles of the alkylation agent that (i) equals or exceeds an amount of moles of tertiary nitrogen atoms in the starting material, or (ii) is less than an amount of moles of tertiary nitrogen atoms in the starting material.

Embodiment 18A. The method of any of Embodiments 14 to 17, wherein the providing of the starting material comprises (i) providing a stream comprising methyl 3-(2-(dimethylamino)ethoxy)propanoate, ethyl 3-(2-(dimethylamino)ethoxy)propanoate, 2-(dimethylamino)ethyl 3-methoxypropanoate, 2-(dimethylamino)ethyl 3-ethoxypropanoate, 2-(dimethylamino)ethyl 3-(2-(dimethylamino)ethoxy)propanoate, 2-(dimethylamino)ethyl acrylate, 2-(dimethylamino)ethan-1-ol, methyl 3-methoxypropanoate, ethyl 3-ethoxypropanoate or a combination thereof,

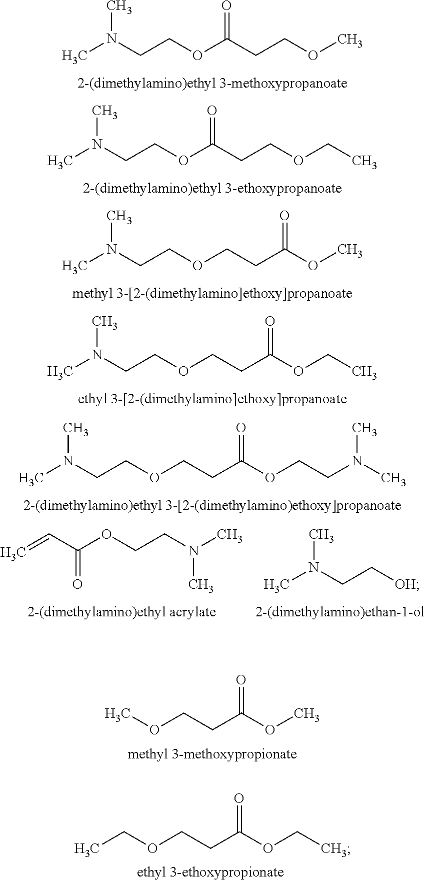

and
(ii) separating from the stream, such as by distillation or rectification, at least a portion of the methyl 3-(2-(dimethylamino)ethoxy)propanoate, at least a portion of the ethyl 3-(2-(dimethylamino)ethoxy)propanoate, at least a portion of the 2-(dimethylamino)ethyl 3-methoxypropanoate, at least a portion of the 2-(dimethylamino)ethyl 3-ethoxy propanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof.

Embodiment 18B. The method of any of Embodiments 14 to 17, Wherein the providing of the starting material comprises (i) providing a stream comprising methyl 3-(2-(dimethylamino)ethoxy)propanoate. 2-(dimethylamino)ethyl 3-methoxypropanoate, 2-(dimethylamino)ethyl 3-(2-(dimethylamino)ethoxy)propanoate, 2-(dimethylamino)ethyl acrylate, 2-(dimethylamino)ethan-1-ol, methyl 3-methoxypropanoate, or a combination thereof,

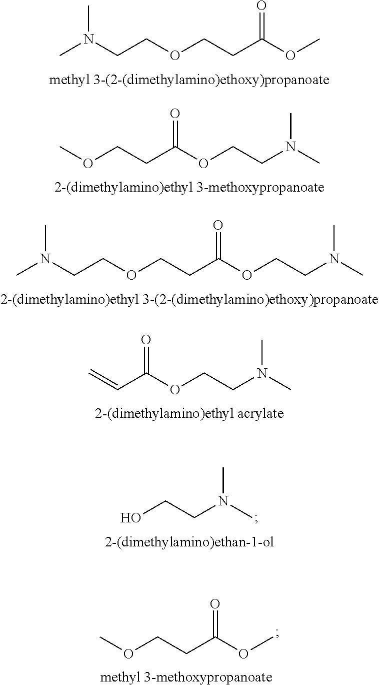

and
(ii) separating from the stream, such as by distillation or rectification, at least a portion of the methyl 3-(2-(dimethylamino)ethoxy)propanoate, at least a portion of the 2-(dimethylamino)ethyl 3-methoxy propanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, or a combination thereof.

Embodiment 19. The method of Embodiment 18, wherein the stream is a waste stream.

Embodiment 20. The method of Embodiment 19, wherein the waste stream is from a process that produces a polymerizable monomer or a precursor thereof, such as an acrylate monomer or a methacrylate monomer.

Embodiment 21(A). The method of any of Embodiments 18 to 20, further comprising converting the at least a portion of the methyl 3-(2-(dimethylamino)ethoxy)propanoate, the at least a portion of the ethyl 3-(2-(dimethylamino)ethoxy)propanoate, the at least a portion of the 2-(dimethylamino)ethyl 3-methoxypropanoate, the at least a portion of the 2-(dimethylamino)ethyl 3-ethoxypropanoate, the at least a portion of the 2-(dimethylamino)ethyl acrylate, or a combination thereof to 2-(dimethylamino)ethyl 3-(2-(dimethylamino)ethoxy)propanoate; wherein, optionally, the converting of the at least a portion of the methyl 3-(2-(dimethylamino)ethoxy)propanoate, the at least a portion of the ethyl 3-(2-(dimethylamino)ethoxy)propanoate, the at least a portion of the 2-(dimethylamino)ethyl 3-methoxypropanoate, the at least a portion of the 2-(dimethylamino)ethyl 3-ethoxypropanoate, the at least a portion of the 2-(dimethylamino)ethyl acrylate, or the combination thereof to 2-(dimethylamino)ethyl 3-(2-(dimethylamino)ethoxy)propanoate comprises contacting (i) the at least a portion of the methyl 3-(2-(dimethylamino)ethoxy)propanoate, the at least a portion of the 2-(dimethylamino)ethyl 3-methoxypropanoate) the at least a portion of the ethyl 3-(2-(dimethylamino)ethoxy)propanoate, the at least a portion of the 2-(dimethylamino)ethyl 3-ethoxypropanoate, the at least a portion of the 2-(dimethylamino)ethyl acrylate, or the combination thereof, and (ii) a base (e.g., KOH), such as a catalytic amount of the base, wherein, optionally, the contacting occurs in a liquid, which may be an aqueous liquid and/or an organic liquid, wherein, optionally, the liquid comprises an alcohol, such as an amino alcohol (e.g., 2-(dimethylamino)ethan-1-ol); and wherein, optionally, at least 40 mol %, at least 50 mol %, at least 60 mol %, at least 70 mol %, or at least 80 mol % of the methyl 3-(2-(dimethylamino)ethoxy)propanoate, at least 40 mol %, at least 50 mol %, at least 60 mol %, at least 70 mol %, or at least 80 mol % of the 2-(dimethylamino)ethyl 3-methoxypropanoate or 2-(dimethylamino)ethyl 3-ethoxypropanoate, and/or at least 40 mol %, at least 50 mol %, at least 60 mol %, at least 70 mol %, or at least 80 mol % of the 2-(dimethylamino)ethyl acrylate is converted to 2-(dimethylamino)ethyl 3-(2-(dimethylamino)ethoxy) propanoate.

Embodiment 21(B). The method of any of Embodiments 18 to 20, further comprising converting the at least a portion of the methyl 3-(2-(dimethylamino)ethoxy)propanoate, the at least a portion of the 2-(dimethylamino)ethyl 3-methoxypropanoate, the at least a portion of the 2-(dimethylamino)ethyl acrylate, or a combination thereof to 2-(dimethylamino)ethyl 3-(2-(dimethylamino)ethoxy)propanoate; wherein, optionally, the converting of the at least a portion of the methyl 3-(2-(dimethylamino)ethoxy)propanoate, the at least a portion of the 2-(dimethylamino)ethyl 3-methoxypropanoate, the at least a portion of the 2-(dimethylamino)ethyl acrylate, or the combination thereof to 2-(dimethylamino)ethyl 3-(2-(dimethylamino)ethoxy) propanoate comprises contacting (i) the at least a portion of the methyl 3-(2-(dimethylamino)ethoxy)propanoate, the at least a portion of the 2-(dimethylamino)ethyl 3-methoxypropanoate, the at least a portion of the 2-(dimethylamino)ethyl acrylate, or the combination thereof, and (ii) a base (e.g., KOH), such as a catalytic amount of the base, wherein, optionally, the contacting occurs in a liquid, which may be an aqueous liquid and/or an organic liquid, wherein, optionally, the liquid comprises an alcohol, such as an amino alcohol (e.g., 2-(dimethylamino)ethan-1-ol); and wherein, optionally, at least 40 mol %, at least 50 mol %, at least 60 mol %, at least 70 mol %, or at least 80 mol % of the methyl 3-(2-(dimethylamino)ethoxy)propanoate, at least 40 mol %, at least 50 mol %, at least 60 mol %, at least 70 mol %, or at least 80 mol % of the 2-(dimethylamino)ethyl 3-methoxypropanoate, and/or at least 40 mol %, at least 50 mol %, at least 60 mol %, at least 70 mol %, or at least 80 mol % of the 2-(dimethylamino)ethyl acrylate is converted to 2-(dimethylamino)ethyl 3-(2-(dimethylamino)ethoxy) propanoate.

Embodiment 22(A). The method of any of Embodiments 18 to 21, wherein (i) the methyl 3-(2-(dimethylamino)ethoxy)propanoate or the ethyl 3-(2-(dimethylamino)ethoxy)propanoate is present in the stream at a concentration of about 1% to about 20%, about 8% to about 20%, or about 10% to about 15%, by weight; (ii) the 2-(dimethylamino)ethyl 3-methoxypropanoate the 2-(dimethylamino)ethyl 3-ethoxypropanoate is present in the stream at a concentration of about 1% to about 60%, about 10% to about 40%, about 15% to about 30%, or about 20% to about 25%, by weight; (iii) the 2-(dimethylamino)ethyl 3-(2-(dimethylamino)ethoxy)propanoate is present in the stream at a concentration of about 5% to about 40%, about 10% to about 40%, about 20% to about 40%, or about 25% to about 35%, by weight; (iv) the 2-(dimethylamino)ethyl acrylate is present in the stream at a concentration of about 1% to about 25%, about 10% to about 20%, or about 12% to about 18%, by weight; (v) the methyl 3-methoxypropanoate or the ethyl 3-ethoxypropanoate is present in the stream at a concentration of about 0.5 to about 2%, or about 0.8% to about 1.2%, by weight; (vi) the 2-(dimethylamino)ethyl acrylate is present in the stream at a concentration of about 1% to about 25%, about 5% to about 20%, or about 10% to about 20%, by weight; or (vii) any combination thereof.

Embodiment 22(B). The method of any of Embodiments 18 to 21, wherein (i) the methyl 3-(2-(dimethylamino)ethoxy)propanoate is present in the stream at a concentration of about 1% to about 20%, about 8% to about 20%, or about 10% to about 15%, by weight; (ii) the 2-(dimethylamino)ethyl 3-methoxypropanoate is present in the stream at a concentration of about 1% to about 60%, about 10% to about 40%, about 15% to about 30%, or about 20% to about 25%, by weight; (iii) the 2-(dimethylamino)ethyl 3-(2-(dimethylamino)ethoxy)propanoate is present in the stream at a concentration of about 5% to about 40%, about 10% to about 40%, about 20% to about 40%, or about 25% to about 35%, by weight; (iv) the 2-(dimethylamino)ethyl acrylate is present in the stream at a concentration of about 1% to about 25%, about 10% to about 20%, or about 12% to about 18%, by weight: (v) the methyl 3-methoxypropanoate is present in the stream at a concentration of about 0.5 to about 2%, or about 0.8% to about 1.2%, by weight; (vi) the 2-(dimethylamino)ethyl acrylate is present in the stream at a concentration of about 1% to about 25%, about 5% to about 20%, or about 10% to about 20%, by weight; or (vii) any combination thereof.

Embodiment 23. The method of any of Embodiments 14 to 22, further comprising subjecting the alkylated material to a hydrolysis reaction to produce a hydrolyzed material.

Embodiment 24. The method of Embodiment 23, wherein the hydrolysis reaction comprises contacting the alkylated material and water, wherein the water has a pH less than 7 or greater than 7, and the pH is effective to achieve ester hydrolysis.

Embodiment 25. The method of Embodiment 24, wherein the alkylated material comprises, consists essentially of, or consists of a compound of formula (III), a compound of formula (IIIa), a compound of formula (IIIb), a compound of formula (IIIc), a compound of formula (IIId), or a combination thereof as defined in any of Embodiments 1 to 13;

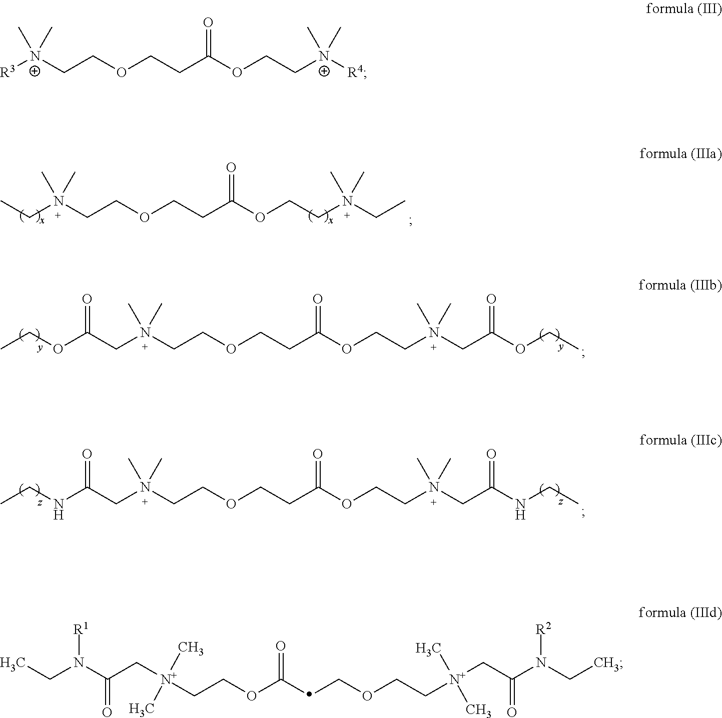

wherein x, y, and z, independently, are 4 to 29 and and R¹, R², independently, are a C₁-C₃₀ hydrocarbyl; and wherein the hydrolyzed material comprises, consists essentially of, or consists of a compound of formula (III′), a compound of formula (III″), a compound of formula (IIIa′), a compound of formula (IIIa″), a compound of formula (IIIb′), a compound of formula (IIIb″), a compound of formula (IIIc′), a compound of formula (IIIc″), a compound of formula (IIId′), a compound of formula (IIId″), or a combination thereof;

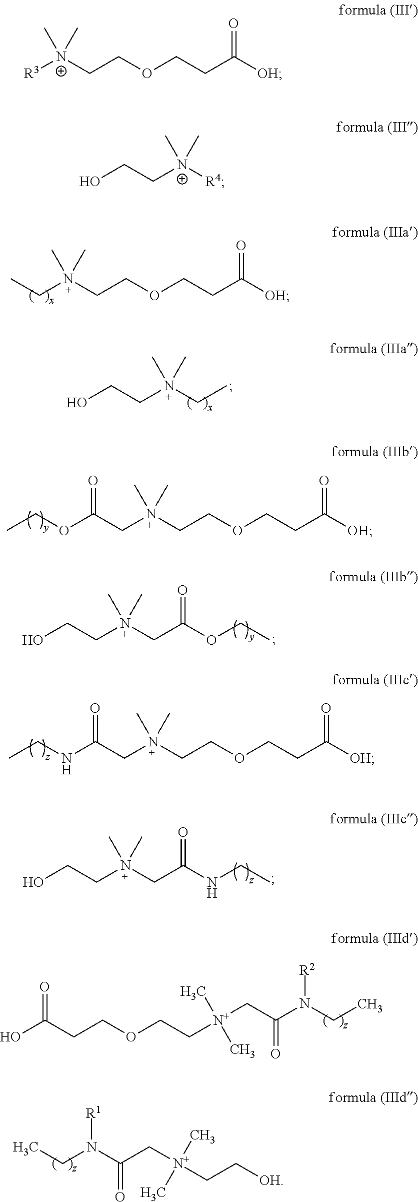

Embodiment 26. The method of any of Embodiments 14 to 25, wherein a product of the method comprises, consists essentially of, or consists of a compound of formula (I), a compound of formula (II), a compound of formula (III), a compound of formula (IV), a compound of formula (V), the compound of formula (VI), the compound of formula (VII), the compound of formula (VIII), one or more hydrolysis products of the compound of formula (I), one or more hydrolysis products of the compound of formula (II), one or more hydrolysis products of the compound of formula (III), one or more hydrolysis products of the compound of formula (IV), one or more hydrolysis products of the compound of formula (V), one or more hydrolysis products of the compound of formula (VI), one or more hydrolysis products of the compound of formula (VII), one or more hydrolysis products of the compound of formula (VIII) or a combination thereof, as defined by any of Embodiments 1 to 13.

1. A method of forming a composition, the method comprising:
(i) providing a starting material comprising methyl 3-(2-(dimethylamino)ethoxy)propanoate, 2-(dimethylamino)ethyl 3-methoxypropanoate, ethyl 3-(2-(dimethylamino)ethoxy)propanoate, 2-(dimethylamino)ethyl 3-ethoxypropanoate, 2-(dimethylamino)ethyl 3-(2-(dimethylamino)ethoxy)propanoate, 2-(dimethylamino)ethyl acrylate, 2-(dimethylamino)ethan-1-ol, or a combination thereof, 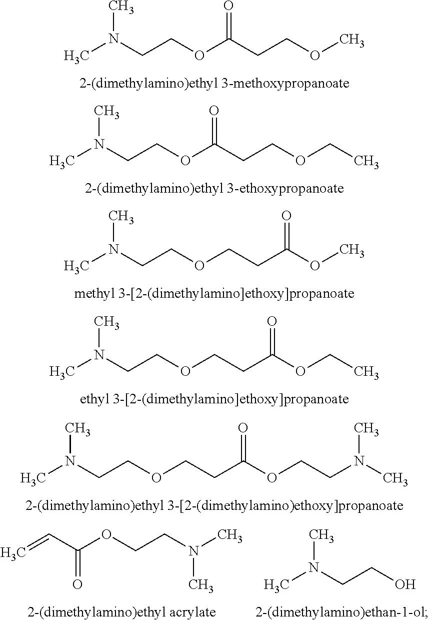
and
(ii) contacting the starting material with an alkylation agent to form an alkylated material.

2. The method claim 1, wherein the providing of the starting material comprises:
(i) providing a waste stream comprising methyl 3-(2-(dimethylamino)ethoxy)propanoate, 2-(dimethylamino)ethyl 3-methoxypropanoate, ethyl 3-(2-(dimethylamino)ethoxy)propanoate, 2-(dimethylamino)ethyl 3-ethoxypropanoate, 2-(dimethylamino)ethyl 3-(2-(dimethylamino)ethoxy)propanoate, 2-(dimethylamino)ethyl acrylate, 2-(dimethylamino)ethan-1-ol, methyl 3-methoxypropanoate, ethyl 3-ethoxypropanoate, or a combination thereof, 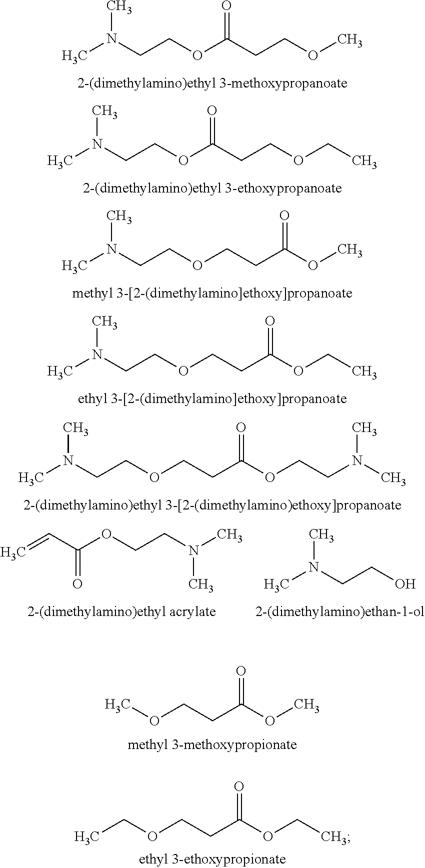
and
(ii) separating from the waste stream at least a portion of the methyl 3-(2-(dimethylamino)ethoxy)propanoate, at least a portion of the 2-(dimethylamino)ethyl 3-methoxypropanoate, at least a portion of the ethyl 3-(2-(dimethylamino)ethoxy)propanoate, at least a portion of the 2-(dimethylamino)ethyl 3-ethoxypropanoate, at least a portion of the 2-(dimethylamino)ethyl 3-(2-(dimethylamino)ethoxy)propanoate, at least a portion of the 2-(dimethylamino)ethyl acrylate, at least a portion of the 2-(dimethylamino)ethan-1-ol, at least a portion of the methyl 3-methoxypropanoate, at least a portion of the ethyl 3-ethoxypropanoate, or a combination thereof.

3. The method of claim 2, wherein the waste stream is from a process that produces a polymerizable monomer or a precursor thereof.

4. The method of claim 2, further comprising converting the at least a portion of the methyl 3-(2-(dimethylamino)ethoxy)propanoate, the at least a portion of the 2-(dimethylamino)ethyl 3-methoxypropanoate, the at least a portion of the ethyl 3-(2-(dimethylamino)ethoxy)propanoate, the at least a portion of the 2-(dimethylamino)ethyl 3-ethoxypropanoate, the at least a portion of the 2-(dimethylamino)ethyl 3-(2-(dimethylamino)ethoxy)propanoate, the at least a portion of the 2-(dimethylamino)ethyl acrylate, the at least a portion of the 2-(dimethylamino)ethan-1-ol, the at least a portion of the methyl 3-methoxypropanoate, the at least a portion of the ethyl 3-ethoxypropanoate, or the combination thereof to 2-(dimethylamino)ethyl 3-(2-(dimethylamino)ethoxy)propanoate;
wherein the converting comprises contacting (i) a base and (ii) the at least a portion of the methyl 3-(2-(dimethylamino)ethoxy)propanoate, the at least a portion of the 2-(dimethylamino)ethyl 3-methoxypropanoate, the at least a portion of the ethyl 3-(2-(dimethylamino)ethoxy)propanoate, the at least a portion of the 2-(dimethylamino)ethyl 3-ethoxypropanoate, the at least a portion of the 2-(dimethylamino)ethyl 3-(2-(dimethylamino)ethoxy)propanoate, the at least a portion of the 2-(dimethylamino)ethyl acrylate, the at least a portion of the 2-(dimethylamino)ethan-1-ol, the at least a portion of the methyl 3-methoxypropanoate, the at least a portion of the ethyl 3-ethoxypropanoate, or the combination thereof; and
wherein at least 40 mol % of the at least a portion of the methyl 3-(2-(dimethylamino)ethoxy)propanoate, at least 40 mol % of the at least a portion of the 2-(dimethylamino)ethyl 3-methoxypropanoate, at least 40 mol % of the at least a portion of the ethyl 3-(2-(dimethylamino)ethoxy)propanoate, at least 40 mol % of the at least a portion of the 2-(dimethylamino)ethyl 3-ethoxypropanoate, at least 40 mol % of the at least a portion of the 2-(dimethylamino)ethyl 3-(2-(dimethylamino)ethoxy)propanoate, at least 40 mol % of the at least a portion of the 2-(dimethylamino)ethyl acrylate, at least 40 mol % of the at least a portion of the 2-(dimethylamino)ethan-1-ol, at least 40 mol % of the at least a portion of the methyl 3-methoxypropanoate, or at least 40 mol % of the at least a portion of the ethyl 3-ethoxypropanoate is converted to 2-(dimethylamino)ethyl 3-(2-(dimethylamino)ethoxy)propanoate.

5. The method of claim 1, wherein the alkylation agent comprises— 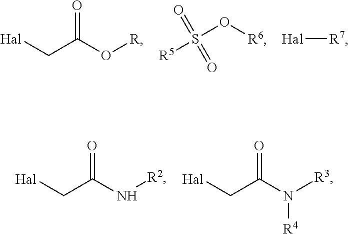
or a combination thereof;
wherein Hal is a halogen atom; and wherein R, R², R³, R⁵, R⁶, and R⁷, independently, are a C₅-C₃₀ hydrocarbyl, and R⁴ is a C₁-C₃₀ hydrocarbyl.

6. The method of claim 1, further comprising subjecting the alkylated material to a hydrolysis reaction to produce a hydrolyzed material.

7. The method of claim 6, wherein the alkylated material comprises a compound of formula (III), and the hydrolyzed material comprises a compound of formula (III′) and a compound of formula (III″): 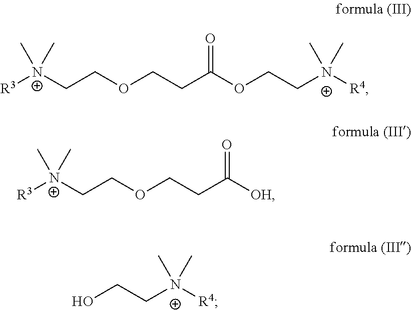
wherein R³ and R⁴ are independently selected from a C₅-C₃₀ hydrocarbyl, formula (a), formula (b), or formula (c); 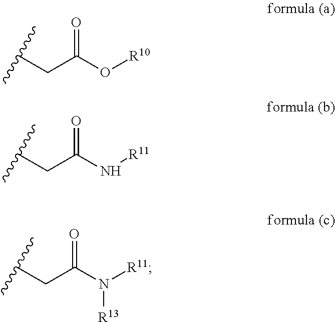
and
wherein R¹⁰ and R¹¹, independently, are a C₅-C₃₀ hydrocarbyl, and R¹² and R¹³, independently, are a C₁-C₃₀ hydrocarbyl.

8. A composition comprising a compound of formula (I), a compound of formula (II), a compound of formula (III), a compound of formula (IV), a compound of formula (V), a compound of formula (VI), a compound of formula (VII), a compound of formula (VIII), or a combination thereof: 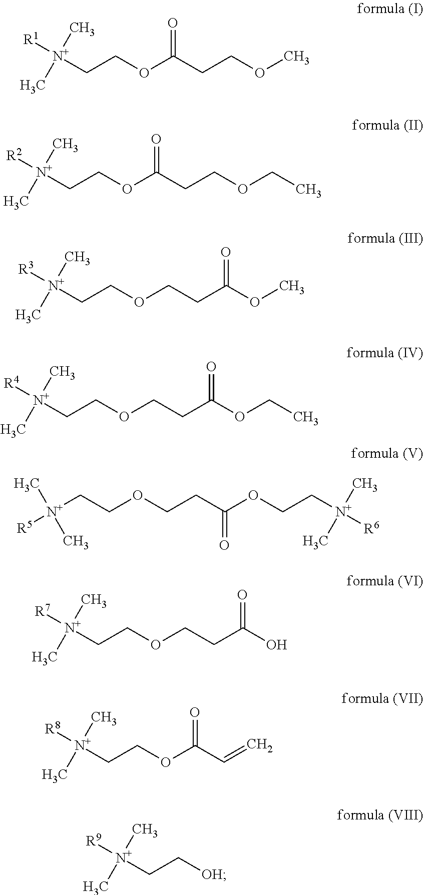
wherein R¹, R², R³, R⁴, R⁵, R⁶, R⁷, R⁸, and R⁹ are independently selected from the group consisting of a C₅-C₃₀ hydrocarbyl, formula (a), and formula (b), and formula (c); 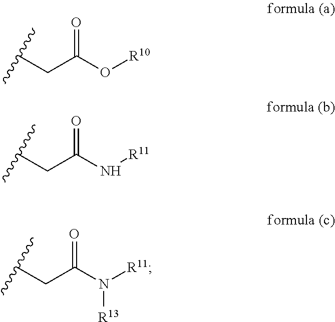
and
wherein R¹⁰ and R¹¹, independently, are a C₅-C₃₀ hydrocarbyl, and R¹² and R¹³, independently, are a C₁-C₃₀ hydrocarbyl.

9. The composition of claim 8, wherein the composition comprises the compound of formula (III), wherein the compound of formula (III) has a structure according to formula (IIIa), formula (IIb), formula (IIIc), or formula (IIId): 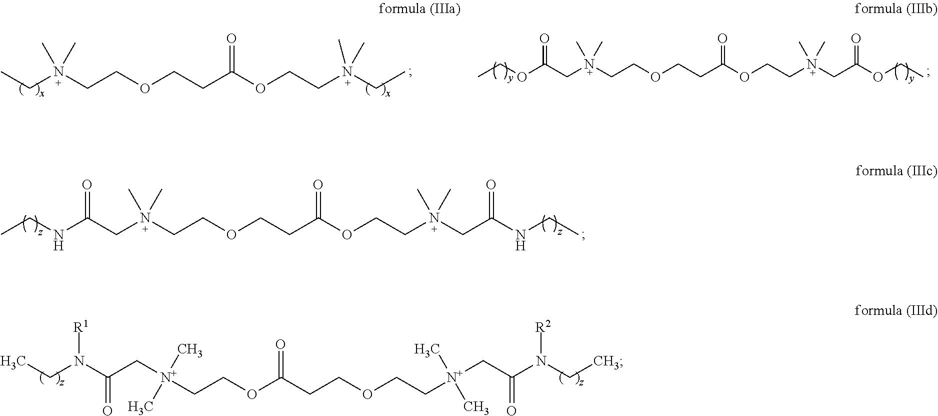
wherein x, y, and z, independently, are 4 to 29, and R¹ and R² independently, are a C₁-C₃₀ hydrocarbyl.

10. The composition of claim 8, wherein the composition comprises the compound of formula (III), and the composition has a critical micelle concentration that is at least 6 times less than a comparative critical micelle concentration of a comparative composition comprising a single-cation surfactant.

11. (canceled)

12. The composition of claim 8, wherein the compound of formula (I), the compound of formula (II), the compound of formula (III), the compound of formula (IV), the compound of formula (V), the compound of formula (VI), the compound of formula (VII), the compound of formula (VIII), or the combination thereof is present in the composition at a concentration of at least 50%, by weight, based on the weight of the composition.

13. The composition of claim 12, wherein the composition is an additive for a cosmetic, a personal care product, a coating, a biocide, a pharmaceutical formulation, an anti-corrosion agent, or an enhanced oil recovery fluid, a fabric softener, a leveling agent, a drying agent, a solubilizer, an emulsifier, a dispersant, a foaming agent, a mining fluid, a waste water treatment fluid, an anti-foaming agent, or an anti-static agent.

14. The composition of claim 8, wherein the compound of formula (I), the compound of formula (II), the compound of formula (III), the compound of formula (IV), the compound of formula (V), the compound of formula (VI), or the combination thereof is present in the composition at a concentration of about 0.01% to about 15% by weight, based on the weight of the composition.

15. The composition of claim 14, wherein the composition is a detergent, a cosmetic, a personal care product, a coating, a biocide, a pharmaceutical formulation, an anti-corrosion agent, an enhanced oil recovery fluid, a fabric softener, a leveling agent, a drying agent, a solubilizer, an emulsifier, a dispersant, a foaming agent, a mining fluid, a waste water treatment fluid, an anti-foaming agent, or an anti-static agent.

16. The method of claim 1, wherein the starting material comprises the 2-(dimethylamino)ethan-1-ol, wherein the starting material is obtained from a waste stream, and wherein the waste stream is from a process that produces a methacrylate monomer.
